(19) United States
(12) Patent Application Publication
(10) Pub. No.: US 2007/0213768 A1
(43) Pub. Date: Sep. 13, 2007

(54) Compositions and Devices Including Same Useful for Anastomosis

(76) Inventors: Irena Wasserman, Rishon Lezion (IL); Dror Michael, Herzlia (IL); David Simchon, Ramat-Efal (IL)

Correspondence Address: Mark M Friedman;Polkinghorn, 9003 Florin Way, Upper Marlboro, MD, 20772, US

(21) Appl. No.: 10/575,951

(22) PCT Filed: Oct. 19, 2004
(86) PCT No.: PCT/IL04/00955

§ 371 Date: Dec. 29, 2006

Related U.S. Application Data
(60) Provisional application No. 60/512,698, filed on Oct. 21, 2003.

(51) Int. Cl. A61B 17/00; C09J 189/00

(52) U.S. Cl. 606/213; 106/158.1; 514/773; 606/214

(57) ABSTRACT

A composition-of-matter is provided. The composition-of-matter comprising a mixture of albumin and a plasticizer. Also provided is a medical devices, a portion of a medical device, a solder or an adhesive composed of such a composition-of-matter and a method of manufacturing same.

### FIELD AND BACKGROUND OF THE INVENTION

The present invention relates to compositions, which can be utilized to fabricate medical devices such as anastomotic devices.

Anastomosis is the surgical joining of two organs. It most commonly refers to a connection which is created between tubular organs, such as blood vessels (i.e., vascular anastomosis) or loops of intestine. Vascular anastomosis is commonly practiced in coronary artery bypass graft surgery (CABG), a surgical procedure which restores blood flow to ischemic heart muscle which blood supply has been compromised by occlusion or stenosis of one or more of the coronary arteries.

Presently, vascular anastomosis is still performed by conventional hand suturing. Suturing, however, has several detrimental aspects. The penetrating needle induces vascular wall damage, which influences the healing response. Suture material is left as an intraluminal foreign body and may cause an inflammatory reaction, thrombocyte aggregation impaired endothelial function, intimal hyperplasia and hence stenosis [see review by Zeebregts (2003) British J. Surg. 90:261-271]. Furthermore, the suturing process is a time consuming and difficult procedure requiring high level of surgical skill. In CABG, for example, the surgeon must have relatively unobstructed access to the anastomosis site within the patient, in order to perform the suturing of the graft to the coronary artery and the blood supplying artery. In less invasive surgical approaches, some of the major coronary arteries including the ascending aorta cannot be easily reached by the surgeon because of their location. This makes suturing either difficult or impossible for some coronary artery sites. In addition, some target vessels, such as heavily calcified coronary vessels, vessels having very small diameter, and previously bypassed vessels may make the suturing process difficult or impossible. Finally, it is important that each anastomosis provides a smooth, open flow path for the blood and that the attachment be completely free of leaks. A completely leak-free seal is not always achieved by hand suturing on the very first try. Consequently, there is a frequent need for re-suturing of the anastomosis to close any leaks that are detected.

For these reasons, non-suture anastomosis is preferred and the development of non-suture techniques is desired.

A first approach to expediting and improving anastomosis procedures is through the use of anastomotic rings for joining blood vessels together. Vascular anastomotic ring techniques primarily include cuffing rings and everting pinned ring devices. These techniques are faster than conventional suturing and have mainly been used to perform end-to-end anastomoses. The main disadvantages of these coupling techniques are the limited number of ring diameters, difficulties due to diameter mismatch and detrimental effects in the perianastomotic area.

A second approach to expediting and improving anastomosis procedures has been through stapling technology. Stapling technology has been successfully employed in many different areas of surgery for making tissue attachments faster and more reliably. The greatest progress in stapling technology has been in the area of gastrointestinal surgery. Various surgical stapling instruments have been developed for end-to-end, side-to-side, and end-to-side anastomoses of hollow or tubular organs, such as the bowel. These instruments, unfortunately, are not easily adaptable for use in creating vascular anastomoses. This is partially due to the difficulty in miniaturizing the instruments to make them suitable for smaller organs such as blood vessels. Possibly even more important is the necessity of providing a smooth, open flow path for the blood. Known gastrointestinal stapling instruments for end-to-side or end-to-end anastomosis of tubular organs are designed to create an inverted anastomosis, that is, one where the tissue folds inward into the lumen of the organ that is being attached. This is acceptable in gastrointestinal surgery, where it is most important to approximate the outer layers of the intestinal tract (the serosa) which is the tissue that grows together to form a strong, permanent connection. However, in vascular surgery this geometry is unacceptable for several reasons. Firstly, the inverted vessel walls would cause a disruption in the blood flow. This could cause decreased flow and ischemia downstream of the disruption, or, worse yet, the flow disruption or eddies (i.e., counterflow) created could become a locus for thrombosis which could shed emboli or occlude the vessel at the anastomosis site. Secondly, unlike the intestinal tract, the outer surfaces of the blood vessels (the adventitia) will not grow together when approximated. The sutures, staples, or other joining device may therefore be needed permanently to maintain the structural integrity of the vascular anastomosis. Thirdly, to establish a permanent, nonthrombogenic vessel, the innermost layer (the endothelium) should grow together for a continuous, uninterrupted lining of the entire vessel.

Other approaches for expediting and improving anastomosis procedures include the use of tubes and stents. Of note, are the absorbable fibrin tubes (made of fibrinogen and thrombin) and the soluble triglyceride based stents [Swenson (1947) Surgery 2:137-143; Moskowitz (1994) Ann; Plast. Surg. 32:612-618]. However, these products are not in use due to the induction of inflammatory responses.

Alternatively anastomosis may be achieved by the use of adhesives (i.e., surgical glues).

At present, the benefits of stents and adhesives hardly overweigh their disadvantages which include toxicity, rigidity, leakage and aneurysm formation (with adhesives) and early occlusion (with stents).

There is thus a widely recognized need for, and it would be highly advantageous to have, compositions, which can be utilized to fabricate medical devices such as anastomosis devices which are devoid of the above limitations.

### SUMMARY OF THE INVENTION

According to one aspect of the present invention there is provided a composition-of-matter comprising a mixture of albumin and a plasticizer.

According to another aspect of the present invention there is provided a medical device, a portion of a medical device, a solder or an adhesive composed of a mixture of albumin and a plasticizer.

According to yet another aspect of the present invention there is provided a method of manufacturing a medical device or a portion of a medical device, the method comprising shaping a mixture of albumin and a plasticizer in a form of the medical device or the portion of the medical device, thereby manufacturing the medical device or the portion of the medical device.

According to further features in preferred embodiments of the invention described below, the medical device is an anastomotic device.

According to still further features in the described preferred embodiments the anastomotic device is selected from the group consisting of a ring, a tube and a stent.

According to still further features in the described preferred embodiments the shaping is facilitated by curing the mixture in a mold.

According to still further features in the described preferred embodiments the curing is effected at a temperature range of 60-90° C.

According to still further features in the described preferred embodiments the curing is effected at a temperature range of 80-90° C.

According to still further features in the described preferred embodiments the curing is effected under conditions such that the medical device generated includes 10-15% water (w/w).

According to still further features in the described preferred embodiments the curing is effected at conditions of 80-95% humidity.

According to still further features in the described preferred embodiments the curing is effected for a duration of 10-120 minutes.

According to still further features in the described preferred embodiments shaping is effected by a method selected from the group consisting of film casting, injection molding, calendaring, compression molding, rotational molding, spin casting and extrusion.

According to still another aspect of the present invention there is provided use of a composition comprising a mixture of albumin and a plasticizer for the manufacture of a medical device, a portion of a medical device, a solder or an adhesive.

According to still further features in the described preferred embodiments the plasticizer is an organic plasticizer.

According to still further features in the described preferred embodiments the organic plasticizer is selected from the group consisting of glycerine, ethylene glycol, polyethylene glycol, 1,2-propane diol, 1,3-propane diol, 1,3 butane diol, 1,4 butane diol, pentaerythritol, glucose and starch.

According to still further features in the described preferred embodiments the organic plasticizer is glycerine.

According to still further features in the described preferred embodiments a ratio of the albumin and the plasticizer in the composition is 3:1 (w/w).

According to still further features in the described preferred embodiments the mixture is in a medical grade.

According to still further features in the described preferred embodiments the mixture is sterile.

The present invention successfully addresses the shortcomings of the presently known configurations by providing compositions, which can be used for generating medical devices.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph illustrating water absorption by thermally cured albumin samples. Water content of cured albumin samples was tested in saline (0.9% NaCl) at 37° C. Note, higher curing temperature resulted in decreased water uptake by the albumin matrix.

FIG. 2 is a graph showing dissolution rate of thermally cured albumin samples. Weight loss of cured albumin samples was tested in saline (0.9% NaCl) at 37° C. Note, curing temperature lower than 80° C. mediated increased dissolution of the sample.

FIG. 3 is a graph showing FTIR spectra of cured (black) and uncured (gray) albumin samples.

FIG. 4 is a photograph showing a number of medical devices manufactured from the compositions of the present invention. Top hat cylinders are photographed over a millimetric page.

### DESCRIPTION OF THE PREFERRED EMBODIMENTS

The present invention is of compositions, which can be used for generating medical devices such as anastomotic devices.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Anastomosis is the surgical joining of two organs, typically tubular organs, such as blood vessels and intestines. The most commonly practiced anastomotic approach is hand suturing, which is relatively inexpensive, reliable, readily available and can be adapted to any tissue condition which may be encountered. Suturing, however, has several drawbacks, such as invasiveness, requirement for high surgical skills and settled unabsorbable suture material which influences the healing response and often leading to excessive inflammation. Each of the newly developed non-suturing approaches and devices, such as rings, stents, tubes and adhesives, has its own inherent disadvantages. These include, rigidity, toxicity, leakage, high cost, reduced strength in larger size vessels, non-compliant anastomosis (as reported with rings), early occlusion (as reported with stents) and demands for surgical skills (as reported with laser tissue welding or soldering).

While reducing the present invention to practice, the present inventors have generated novel albumin based compositions, which can be used for fabricating anastomotic implants and adhesives free of the limitations described above.

In sharp contrast to previously described albumin based compositions such as that used to fabricate the stent described by Xie (2003) Lasers Surg. Med. 294-8, the compositions of the present invention include a combination of albumin and plasticizers, which renders devices generated therefrom less brittle and more flexible while still maintaining mechanical strength for a significant period prior to being resorbed in physiological fluids.

Unlike fibrin compositions which induce allergy and inflammation, the albumin compositions of the present invention are biocompatible (i.e., do not produce toxic or immunological responses in living tissues) and as such are optimal for medical applications.

Thus, according to one aspect of the present invention there is provided a composition-of-matter, which includes a mixture of albumin and a plasticizer.

As used herein “albumin” refers to a 60 kDa water soluble serum protein (e.g., bovine serum albumin and ovalbumin). Commercially available bovine serum albumin can be purchased as a powder from ICN (Shelton Conn. USA, Cat. No. 160069).

As used herein a “plasticizer” refers to a compound, which increases flexibility and reduces stiffness of compositions, such as plastics. Typical plasticizers are low molecular weight organic or inorganic molecules (e.g., low molecular weight polymers). Examples of organic plasticizers include, but are not limited to, glycerine, ethylene glycol, polyethylene glycol, 1,2-propane diol, 1,3-propane diol, 1,3 butane diol, 1,4 butane diol, pentaerytbritol, glucose, starch and a combination thereof According to presently known embodiments of the present invention the plasticizer is preferably glycerine. Preferably, the ratio between albumin and glycerine in the composition of the present invention is 3:1 (w/w).

Since the composition of the present invention and devices fabricated therefrom are utilized in therapeutic applications, the components thereof (e.g., albumin and plasticizer) are preferably sterile (i.e., free of living organisms such as bacteria and yeast) and are of high purity, more preferably medical grade purity (i.e., safe for administration) and even more preferably implant grade purity (i.e., safe for implantation).

It will be appreciated that the composition of the present invention may include other ingredients as well. Such ingredients are preferably biocompatible for use in biological research and medical applications, essentially not producing a toxic, injurious, or immunological response in living tissues. Examples of such ingredients include, but are not limited to, antioxidants, such as vitamin E, which can be used as a biocompatible antioxidant Other suitable ingredients include dyes, such as “Green GLS Dye” (available from Clarian Corp., Charlotte, N.C.) that can be added to facilitate the ability to visualize delivery of the implant composed of the composition of the present invention to the desired site. Preferred dyes are stable to change in the course of sterilization, e.g., by irradiation such as γ or Electron-beam.

Optionally, fillers, such as calcium carbonate, titanium dioxide or barium sulfate can be added as well, in about 0.5% to about 20% (by weight) to affect the viscosity and thixotropic properties of the resultant mixture.

Other components which may be added to the composition of the present invention include reinforcement additives which may be added to improve physical strength (e.g., pyrolitic carbon which is characterized by extraordinary biocompatibility), lubricants which may be added to improve processing (e.g., aqueous solutions of carbohydrates e.g., chondroitin sulphate) and ultraviolet protectors.

The composition of the present invention is prepared by mixing techniques, such as that described in Example 1 of the Examples section which follows.

As mentioned hereinabove, the composition of the present invention can be used as an adhesive or a solder it can be used to fabricate a medical device such as an anastomotic device or a portion thereof.

As used herein the term “adhesive” refers to a substance, which can be utilized to join two surfaces (e.g., tissue surfaces).

As used herein a “solder” refers to a low melting point substance, which can be used in numerous joining applications such as to join two tissue surfaces.

As used herein “a medical device” refers to an instrument, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in man or other animals. Preferably, the medical device of the present invention is an implantable device used for anastomosis (i.e., an anastomotic device such as described in details in the Background section).

In order to generate the medical device, the composition of the present invention is shaped into the form desired. For example, in order to generate sleeves which can be used in vascular anastomosis, the composition of the present invention is subjected to film casting, shaping to a desired form (e.g., cylinder) and thermal curing as is described in detail in Example 1 of the Examples section.

As used herein “film casting” refers to depositing a layer of the composition of the present invention onto a surface, allowing it to solidify, and removing the film thus formed from that surface.

As used herein “curing” is the process by which the physical properties of a material are changed into a more stable and usable condition. This is accomplished by the use of heat (i.e., thermal curing), radiation or reaction with chemical additives.

According to presently known embodiments of the present invention, thermal curing is effected at a temperature range of 60-90 ° C., more preferably 70-90 ° C., even more preferably 80-90° C.; at humidity conditions of 80-95; and for a duration of 10-120 minutes, more preferably 30-120 minutes, even more preferably 60-120 minutes.

Preferably curing is effected so as to maintain water (trace water) concentration in the medical device of 7-15% (e.g., 10-15%).

Medical devices generated using the above-described conditions are characterized by low solubility, improved tensile strength and slow dissolution rate as compared to albumin alone, rendering the devices of the present invention optimal for implantion (see Example 2 of the Examples section which follows). However, since medical devices, adhesives or solders of the present invention are subjected to dissolution when in physiological environment, they are preferably used for applications which require only temporary presence thereof in the site of interest (e.g., anastomosis).

Other suitable techniques which can be used for fabricating the medical device of the present invention include extrusion processes such as ram extrusion; polymeric casting techniques such as solvent casting and spin casting, molding techniques such as blow molding, injection molding and rotational molding and calendaring. Other thermoforming techniques useful with polymeric materials may be employed and chosen to best serve the type of material used and specific characteristics desired.

Following is a short description of processing techniques which may be used for generating the medical devices of the present invention.

Compression molding—A process in which a polymer is molded in a confined shape by applying pressure and usually heat.

Transfer molding—A process in which heat and pressure are used to transfer partially melted polymer into the cavity with a sprue. After the polymer is cured inside the cavity, it is removed. The differences between the compression molding and transfer molding is that the curing time for the latter is usually shorter. The loading time is also shorter. Transfer molding is usually the preferred method for producing parts that have a large variation in section thickness. Cost considerations favor compression molding.

Injection molding—A process whereby a polymer is injected into the mold where it solidifies. When compared to compression molding, injection molding has a shorter molding cycle. This makes injection molding cost cheaper since an equal production amount can be achieved with fewer cavities in the mold. Metal inserts such as bearings, contacts or screws can be inserted into the mold so that they can be molded into the molded parts.

Blow molding—A process, which is used to make thin hollow containers. The rational is to use air pressure to force the material against the mold surfaces. Caution must be made to properly vent the machine to avoid poor surface finish.

Cold molding—The process of compression molding involving shaping the unheated compound in a mold under pressure then heating the article to cure it.

Extrusion—A process whereby the polymer is extruded through dies into a simple shape of any length. The raw composition is fed into the hopper and forced into a heated chamber by a spiral screw. After passing through the die, the viscous material is cooled by air, water or by contact with a cool surface. This process is mostly applicable for producing tubes.

Co-extrusion—The process of combining two or more layers of extrudate to produce a multiple layer product in a single step.

Examples of devices, adhesives and additives which can be produced using the compositions of the present invention and uses thereof are listed in Table 1, below.

TABLE 1

| | Uses |
|---|---|
| Stents | Scaffolds for intraluminal end to end |
| | anastomoses: |
| | Gastrointestinal anastomoses. |
| | Vascular surgery. |
| | Transplantations (heart, kidneys, pancreas, |
| | lungs) |
| | Pulmonary airways (trachea, lungs etc.) |
| | Laser bonding (replacing sutures, clips and |
| | glues. No mechanical approximating device |
| | used during laser bonding) Supporting stents |
| | for keeping body orifices open |
| Sleeves | With or without intraluminal stents. |
| | Outside scaffold for nerves and tendon |
| | anastomoses (suturless and non sutureless) |
| Films | Protective and reinforcing films (for laser |
| | bonding or other glues) |
| | Biodegradable wound dressing |
| | Substrate for cell culturing |
| | Prevention of surgical adhesions |
| | Abdominal wall surgical reinforcement |
| Adhesives (glues) | Reinforced glue for laser bonding |
| | Reinforced sealants |
| Therapeutic agent | For the delivery of drugs such as antibiotics, |
| delivery systems | tissue growth factors and anticoagulants |
| Scaffolds | For tissue engineering |
| Coating | Coating for medical devices or parts thereof, |
| | rendering them more biocompatible |

As set forth above, compositions of the present invention can be used to manufacture medical devices or parts thereof, such as for example, endoprostheses. An examples of a medical device which can be produced is a stent [e.g., for billiary stenting www.amershamnhealth.com/medcyclopaedia/medical/volume%20I/BILIARY%20STE NTING.ASP; Raijman, I., “Biliary and pancreatic stents,” Gastrointest.Endosc.Clin.N.Am., vol. 13, no. 4, pp. 561-viii, 2003; and for reducing fluid pressure, such as on the eye (Glaucoma www2.tau.ac.il/ramot/project.asp?cnumber=1156&title=An%20Innovative%20Impla nt%20for%20Treating%20Glaucoma&word=)]. Such stents can be manufactured by extruding the composition of the present invention into filaments having the desired diameter. The filaments can then be used for making a mesh of the desired texture. The mesh can be used for making stents of desired dimensions (diameter and length). The tube can then be dried and stored Prior to implantation the tube is preferably slightly wetted and then applied with the appropriate introducer.

Examples of other medical devices which can be produced using the compositions of the present invention include, but are not limited to, catheters, guidewires, introducer sheaths, sutures, mesh and the like (see FIG. 4). Procedures for placing such medical devices in the site of interest are well known in the art [see e.g., Atlas of Surgical Operations. Zollinger R M Jr. (1988) Macmillan Publishing Co. pages 36-106; and Friedrich (2002) Circulation 106:I-1]. The compositions of the present invention can also be used for coating such devices, as they impart increased bio-compatibility to one or more surfaces thereof.

Furthermore, when the composition of the present invention includes a therapeutic agent (see further details hereinbelow), specific therapeutic effects can be imparted to the surfaces of such devices. Thus, any medical device to which the bioresorbable coating composition of the present invention can adhere may be used for purposes of the present invention.

Alternatively, compositions of the present invention can be used in tissue engineering applications as supports for cells. Appropriate tissue scaffolding structures are known in the art (see U.S. Pat. No. 6,316,522 and references therein). Methods of seeding and/or culturing cells in tissue scaffoldings are known in the art, such as those methods disclosed in EPO 422 209 B1, WO 88/03785, WO 90/12604, and WO 95/33821 (all hereby incorporated by reference herein).

In a semi solid state (e.g., 50% w/v, film) compositions of the present invention can be used as glues such as for welding (with either thermal or laser energy) or soldering as described in Simhon, D., Brosh, T., Halpern, M., Ravid, A., Vasilyev, T., Kariv, N., Katzir, A., and Nevo, Z., “Closure of skin incisions in rabbits by laser soldering: I: Wound healing pattern,” Lasers Surg. Med., vol. 35, no. 1, pp. 1-11, (2004); Simhon, D., Ravid, A., Halpern, M., Cilesiz, I., Brosh, T., Kariv, N., Leviav, A., and Katzir, A., “Laser soldering of rat skin, using fiberoptic temperature controlled system,” Lasers Surg. Med., vol. 29, no. 3, pp. 265-273, (2001); Zeebregts (2003) British J. Surg. 90:261-271 and references therein.

Compositions of the present invention may also function as delivery vehicles for therapeutic agents. Since the compositions of the present invention are biodegradable they may be used for slow releasinf of therapeutic agents bound thereto, thus serving as a slow release reservoir. As used herein a therapeutic agent refers to any substance having a desired therapeutic effect. Examples of therapeutic agents, include genetic therapeutic agents, non-genetic therapeutic agents, and cells, which can be used in conjunction with the compositions of the invention.

Exemplary non-genetic therapeutic agents include: anti-thrombic agents such as heparin and heparin derivatives; anti-inflammatory agents such as dexamethasone, prednisolone, corticosterone, budesonide, estrogen, sulfasalazine and mesalamine; antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies (e.g., capable of blocking smooth muscle cell proliferation), and thymidine kinase inhibitors; anesthetic agents such as lidocaine, bupivacaine and ropivacaine; anti-coagulants such as D-Phe-Pro-Arg chloromethyl ketone, heparin, hirudin, antithrombin compounds, tissue plasminogen activator (TPA), platelet receptor antagonists, anti-thrombin antibodies, anti-platelet receptor antibodies, aspirin, prostaglandin inhibitors, platelet inhibitors and tick antiplatelet peptides; growth factors; transcriptional activators, and translational promoters; cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins); cholesterol-lowering agents; antibiotics such as triclosan, cephalosporins, aminoglycosides and nitrofurantoin; anti viral agents (e.g., Ganciclovir and interferon), cytotoxic agents, cytostatic agents and cell proliferation affectors; vasodilating agents; and agents that interfere with endogenous vascoactive mechanisms.

Examples of genetic therapeutic agents include, but are not limited to, anti-sense DNA and RNA; and DNA coding for: anti-sense RNA, tRNA or rRNA to replace defective or deficient endogenous molecules, and DNA encoding for the above proteins and peptides. Vectors of interest for delivery of genetic therapeutic agents include plasmids; viral vectors such as adenovirus (AV); adenoassociated virus (AAV) and lentivirus; and non-viral vectors such as lipids, liposomes and cationic lipids.

Examples of cells include mammalian cells such as those of human origin (autologous or allogeneic), including stem cells, or from an animal source (xenogeneic), all of which can be genetically engineered to deliver proteins of interest.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology. A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. Y., Eds. (1984); “Animal Cell Culture” Freshney, R. L, ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Fabrication of Albumin Tubes

Materials—Albumin—Bovine serum albumin solid powder was purchased from ICN (Cat #160069). Glycerol (Glycerine)—analytical grade was purchased from Frutarom (Cat #551190). Pentaerythritol—analytical grade was purchased from Aldrich Chemicals (Cat # P475-5, Sigma-Aldrich, St. Louis, Mo.). Ethylene glycol C.P., was purchased from Frutarom (Cat #5551530). All other chemicals were at least 98% pure and purchased from Aldrich (Sigma-Aldrich, St. Louis, Mo.).

Film casting—Albumin films were cast from an aqueous solution of albumin and glycerine. The solution was prepared by magnetically stirring water (6.4 g), glycerol (0.9 g) and albumin (2.7 g). The solution was cast on teflon surfaces and allowed to settle until a homogeneous film was formed (room temperature for 15-25 hrs depending on the film thickness). The substrate was leveled so as to obtain films of uniform thickness. Thereafter drying was effected in a dessicator for 0.5-1 hr in the presence of silica gel which served as a drying agent. To prevent the formation of air bubbles which result in imperfections in the film, vacuum of 200-300 mm Hg was applied for several minutes until all bubbles popped. Thereafter air was re-introduced into the dessicator. The thickness of the films was a function of the volume that was cast as well as the shape of the mold and the concentration of the solution.

Sleeve shaping—Tubes were prepared from dry films that were rewetted shortly before wrapping them around a cylinder made of glass to obtain a flexible and sticky composition (the edge is attached or glued by overlapping and the stickiness is used for adhesion). Drying was accomplished while the albumin was on the mold (room temperature for several minutes). The dried tube was then pulled off the cylinder. The inner diameter (ID) of the tube (outer diameter of the cylinder) ranged between 1-3 mm, dependent on the outer diameter of the glass tube.

Curing—Thermal curing of the tubes was effected to obtain a product with desired features. A number of temperature conditions were applied including 74, 77, 80, 81 and 85 ° C. The duration of curing varied from 30-120 min as well as the trace water and the of humidity of the atmosphere in which the curing was carried out.

### Example 2

### Albumin Sleeves Generated According to the Teachings of the Present Invention are Tensile and Stable Exhibiting Limited Water Absorption and Weight Loss

Characterization of cured albumin samples generated as described in Example 1, included water absorption, rate of weight loss during immersion in saline and tensile properties.

Experimental Procedures

Water absorption and weight loss—Water absorption and weight loss during immersion were tested in saline (0.9% NaCl) at 37° C. A series of identical samples was immersed and pulled out at different time points from the immersion tank. The samples were wiped, weighed and then dried to constant weight which allowed determining both weight loss and water absorption.

Tensile properties—Tensile properties were tested on films that were cut into ‘dog bone’ shapes such that the central section was 50.0 mm long, 5 mm wide and 0.1 mm thick. Films tested were all of the same initial composition yet differed as follows:

(i) Dry films which were not thermally cured

(ii) Dry films which were thermally cured (85° C. for 2 hrs).

(iii) As in (ii) yet wetted (soaked in water) for 2 hours at 37° C. prior to being subjected to tensile testing.

Samples were glued to the clamps of an Instron machine using cyanoacrylate glue or epoxy and were tested according to art guidelines (see e.g., http://www.instron.com/applications/test_types/tension/index.asp).

Results

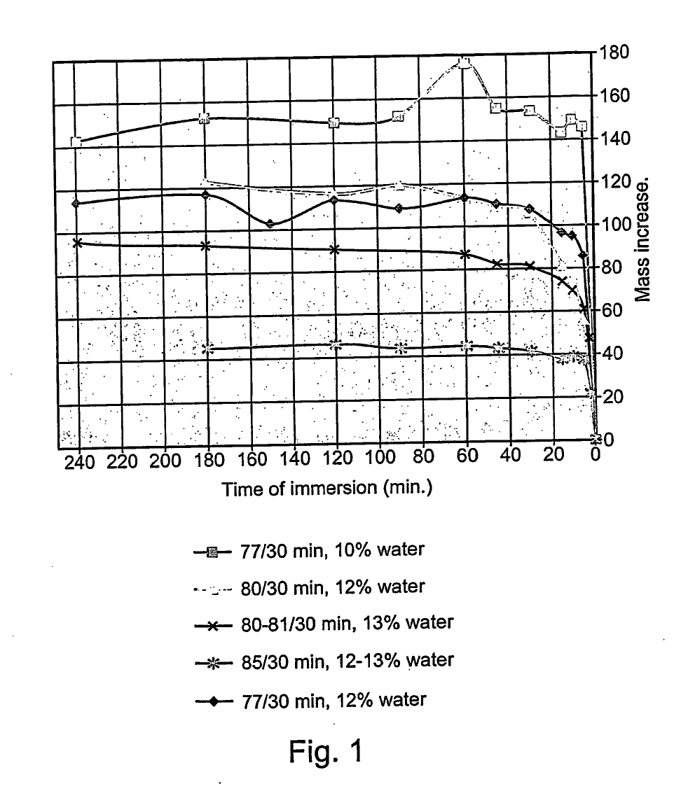

Water absorption of samples including glycerine and 10-13% water which were cured at different temperature conditions is shown in FIG. 1. Evidently, the higher the curing temperature, the lower the water uptake capability of the albumin matrix was. Furthermore, most of the water uptake occured in the first 20 minutes if immersion in saline. Notably, curing of albumin films containing no glycerine did not change their water solubility and consequently these films completely dissolved in saline (data not shown).

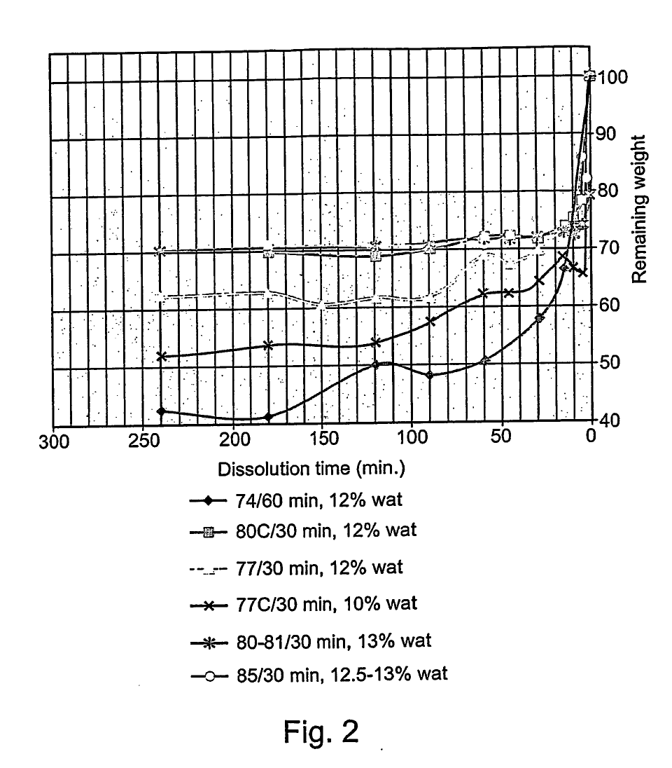

Dissolution rate of cured albumin samples in saline is depicted in FIG. 2. Rapid weight loss was seen at the first 20 minutes of incubation which was probably due to glycerol diffusion out of the samples. Samples that were cured at temperatures lower than 80° C. showed also dissolution of some of the albumin matrix. Most of the dissolution in saline occurred during the initial 2 to 3 hours.

Results of tensile properties analysis are presented in Table 2, below.

TABLE 2

| | | | Dry films | | Dry films which |
|---|---|---|---|---|---|
| | Dry films | | which were | | were thermally |
| | which were not | | thermally | | cured (85° C. for |
| | thermally | | cured (85° C. | | 2 hrs) and rewetted |
| | cured | | for 2 hrs) | | prior to analysis |
| Young's modulus | 140 | (25) | 240 | (25) | 1.5 (0.3) |
| (MPa) | | | | | |
| Tensile Strength | 4.5 | (0.6) | 6.9 | (0.5) | 1.3 (0.3) |
| (MPa) | | | | | |

* all values are given in Mega Pascal with SD in brackets

As is evident from Table 2 above, cured samples exhibited enhanced strength as compared to non-cured samples. These results indicate that real physical changes have occurred while curing (probably some entanglement of polymeric chains).

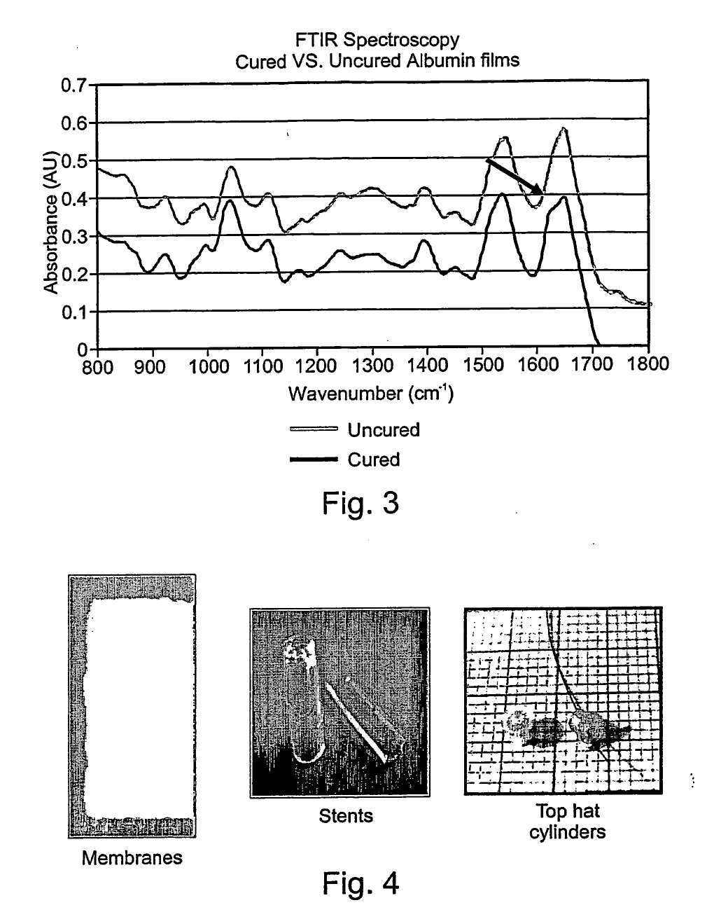

Samples of albumin films were also characterized using Fourier transform infrared (FTIR) spectroscopy. FIG. 3 compares FTIR spectra of thermally cured and uncured films. Evidently, the spectra showed a difference in the Amide I peak, which is indicated by a black arrow pointing to the shoulder that was present in the cured samples but was absent from the uncured material.

Animal studies carried out on a rabbit model demonstrated that albumin stents generated according to the teachings of the present invention maintain adequate mechanical properties during surgery and are resorbable in the time range of 24-48 hrs (not shown, Simhon, D., Kopelman, D., Hashmonai, M., Veserman, I., Dror, M., Vasilyev, T., Halpern, M., Kariv, N., and Katzir, A, “End-To-End Small Bowel Anastomosis by Temperature Controlled CO₂ Laser Soldering and an Albumin Stent—a Feasibility Study.” SPIE Proceedings 2004, Vol. 5312, pp. 177-84). These results suggest that such a sleeve that is implanted by the surgeon in the intestines provides adequate mechanical support to the vessel walls for at least several hours. At the same time, this sleeve is also resorbed in a time period that depends on the curing conditions. It appears that some proteolytic enzymes are responsible for dissolution of samples that remained intact in saline.

Thus, heat curing of albumin films which included a plasticiser changes the solubility, water uptake and tensile properties of albumin. This suggests that the presence of the plasticizers makes a difference as curing of unplasticized albumin resulted in totally soluble specimen. The curing of plasticized specimen on the other hand resulted in lower solubility of the film, lower water uptake and improved tensile properties. It is believed that non-reversible secondary denaturation of albumin occurs during curing, rather than covalent cross-linking.

The present results indicate that the higher the curing temperature and the longer the duration of curing, the lower the solubility of the film and the lower its interaction with water. While the solubility is decreasing, also the water uptake or swelling decreases. These two properties are highly significant for the application of devices made by this novel process. Furthermore, the samples that demonstrated reduced swelling were also less flexible and when implanted may retain mechanical strength for a longer time. In the physiological environment, the plasticizer may diffuse out of the device allowing enzymes to penetrate the matrix thereby taking part in a slow degradation and resorption. The fact that samples that did not dissolve in saline at physiologic conditions, disappeared following 24 hrs in the animal supports this hypothesis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

What is claimed is:

1. A composition-of-matter comprising a mixture of albumin and a plasticizer.

2. The composition of claim 1, wherein said plasticizer is an organic plasticizer.

3. The composition-of-matter of claim 2, wherein said organic plasticizer is selected from the group consisting of glycerine, ethylene glycol, polyethylene glycol, 1,2-propane diol, 1,3-propane diol, 1,3 butane diol, 1,4 butane diol, pentaerytbritol, glucose and starch.

4. The composition-of-matter of claim 2, wherein said organic plasticizer is glycerine.

5. The composition-of-matter of claim 1, wherein a ratio of said albumin and said plasticizer is 3:1 (w/w).

6. The composition-of-matter of claim 1, wherein the composition-of-matter is in a medical grade.

7. The composition-of-matter of claim 1, wherein the composition-of-matter is sterile.

8. A medical device, a portion of a medical device, a solder or an adhesive composed of a mixture of albumin and a plasticizer.

9. The medical device, the portion of the medical device, the solder or the adhesive of claim 8, wherein said plasticizer is an organic plasticizer.

10. The medical device, the portion of the medical device, the solder or the adhesive of claim 9, wherein said organic plasticizer is selected from the group consisting of glycerine, ethylene glycol, polyethylene glycol, 1,2-propane diol, 1,3-propane diol, 1,3 butane diol, 1,4 butane diol, pentaerythritol, glucose and starch.

11. The medical device, the portion of the medical device, the solder or the adhesive of claim 9, wherein said organic plasticizer is glycerine.

12. The medical device, the portion of the medical device, the solder or the adhesive of claim 8, wherein a ratio of said albumin and said plasticizer in the composition is 3:1 (w/w).

13. The medical device, the portion of the medical device, the solder or the adhesive of claim 8, wherein the medical device is an anastomotic device.

14. The medical device, the portion of the medical device, the solder or the adhesive of claim 13, wherein said anastomotic device is selected from the group consisting of a ring, a sleeve and a stent.

15. The medical device, the portion of the medical device, the solder or the adhesive of claim 8, wherein said mixture is in a medical grade.

16. The medical device, the portion of the medical device, the solder or the adhesive of claim 8, wherein said mixture is sterile.

17. A method of manufacturing a medical device or a portion of a medical device, the method comprising shaping a mixture of albumin and a plasticizer in a form of the medical device or the portion of the medical device, thereby manufacturing the medical device or the portion of the medical device.

18. The method of claim 17, wherein said plasticizer is an organic plasticizer.

19. The method of claim 18, wherein said organic plasticizer is selected from the group consisting of glycerine, ethylene glycol, polyethylene glycol, 1,2-propane diol, 1,3-propane diol, 1,3 butane diol, 1,4 butane diol, pentaerytritol, glucose and starch.

20. The method of claim 18, wherein said organic plasticizer is glycerine.

21. The method of claim 17, wherein a ratio of said albumin and said plasticizer is 3:1 (w/w).

22. The method of claim 17, wherein the medical device is an anastomotic device.

23. The method of claim 22, wherein said anastomotic device is selected from the group consisting of a ring, a tube and a stent.

24. The method of claim 17, wherein said shaping is facilitated by curing said mixture in a mold.

25. The method of claim 24, wherein said curing is effected at a temperature range of 60-90° C.

26. The method of claim 24, wherein said curing is effected at a temperature range of 80-90° C.

27. The method of claim 24, wherein said curing is effected under conditions such that the medical device generated includes 10-15% water (w/w).

28. The method of claim 24, wherein said curing is effected at conditions of 80-95% humidity.

29. The method of claim 24, wherein said curing is effected for a duration of 10-120 minutes.

30. The method of claim 24, wherein shaping is effected by a method selected from the group consisting of film casting, injection molding, calendaring, compression molding, rotational molding, spin casting and extrusion.

31. The method of claim 17, wherein said mixture is in a medical grade.

32. The method of claim 17, wherein said mixture is sterile.

33. Use of a composition comprising a mixture of albumin and a plasticizer for the manufacture of a medical device, a portion of a medical device, a solder or an adhesive.

34. The use of claim 33, wherein said plasticizer is an organic plasticizer.

35. The use of claim 34, wherein said organic plasticizer is selected from the group consisting of glycerine, ethylene glycol, polyethylene glycol, 1,2-propane diol, 1,3-propane diol, 1,3 butane diol, 1,4 butane diol, pentaerythritol, glucose and starch.

36. The use of claim 34, wherein said organic plasticizer is glycerine.

37. The use of claim 33, wherein a ratio of said albumin and said plasticizer in the composition is 3:1 (w/w).

38. The use of claim 33, wherein said mixture is in a medical grade.

39. The use of claim 33, wherein said mixture is sterile.
